(19) United States
(12) Patent Application Publication
(10) Pub. No.: US 2003/0199577 A1
(43) Pub. Date: Oct. 23, 2003

(54) Substrate with controlled amine density and regular spacing and method for preparing the same

(76) Inventors: Joon-Won Park, Pohang-city (KR); Jeo-Young Shim, Masan-city (KR); Joong-Ho Moon, Changwon-city (KR); Bong-Jin Hong, Pohang-city (KR)

Correspondence Address: BAKER & BOTTS, 30 ROCKEFELLER PLAZA, NEW YORK, NY, 10112

(21) Appl. No.: 10/363,948

(22) PCT Filed: Sep. 5, 2001
(86) PCT No.: PCT/KR01/01501

(30) Foreign Application Priority Data
Sep. 5, 2000 (KR) 2000/52504

(51) Int. Cl. A61K 31/325; C07C 271/20

(52) U.S. Cl. 514/489; 560/159

(57) ABSTRACT

The present invention relates to a substrate useful to bio-chips comprising a molecular layer having low surface density of amines, and it provides a compound of Chemical Formula (1) represented by N—CBZ-[1]amine-[9]acid having a carboxylic acid, a substrate comprising a surface having a molecular layer prepared by reacting amine groups of aminosilylated surface of the substrates with a compound of Chemical Formula (1) having a cone shape, and methods for preparing the same.

### CROSS REFERENCE TO RELATED APPLICATION

This application is based on application No. 2000-52504 filed in the Korean Industrial Property Office on Sep. 5, 2000, the content of which is incorporated hereinto by reference.

### BACKGROUND OF THE INVENTION

1. (a) Field of the Invention

The present invention relates to a substrate that is applicable to a biochip, having a molecular layer including an amine group with a low density on its surface, and more particularly to a compound used for formation of a molecular layer with a low density, and a method of preparing the same, and a substrate including a molecular layer having an amine group prepared from the compound, and a method of preparing the same.

2. (b) Description of the Related Art

Silylation on a substrate surface, and in particular aminosilylation, has been applied to various processes such as fixation of bio-molecules like enzymes and an antibodies; inorganic catalyst fixation; modification of electrolyte; chromatography; and a building formation of various types of molecules having ionic polymers, optical nonlinear chromophoric groups, fullerene, porphyrin, and complex and inorganic colloidal silica for self-assembly. The chemical and physical properties of an aminosilane layer formed on a substrate are critical factors in a molecular structure and a surface density of self-assembly molecules, and in a structure and properties of a functional film formed on the substrate.

When amine groups are formed on a solid substrate, 1 to 10 amines per 100 Å² are needed. A solid substrate having amine groups on the surface is used for biochip or DNA chip boards. However, when DNA oligonucleotides or other bio-molecules are fixed on the substrate comprising 1 to 10 amines per 100 Å², a high steric hindrance stresses the molecules, so that the molecules are not fixed. In addition, a DNA chip needs to have a sufficient space among the fixed molecules in order to hybridize the DNA and to increase the chip efficiency. To solve these problems, Talov et al. suggested that density can be controlled by decreasing the concentration of self-assembly molecules (J. Am. Chem. Soc. 120, 9787(1998)). However, the surface is controlled indirectly, and the distribution is not regular since molecules having functional groups agglutinate with each other. In other words, the regular spacing of DNA is difficult to control. Therefore, optimal hybridization efficiency and concentration are critical, but a concentration greater than the determined concentration of DNA is difficult to apply.

As another example, Okahata et al. suggested that DNA be introduced on a substrate surface by bonds of Biotin and Avidin (J. Am. Chem. Soc. 120, 8537(1998)). The method is as follows: gold is deposited on a QCM (Quartz Crystal Microbalance) surface to prepare a spacer having thiol and biotin, and DNA having biotin as an end is introduced to the QCM. In this method, a QCM frequency signal is changed as the hybridization proceeds. However, the method in which the biotin-abidin bond is used is indirect, and it is limited by use of the QCM, and space is needed proteins for formation of the helical structure as well as DNA.

Whitesell et al. suggested that a single layer of aminotrithiol be piled on a gold surface so that polyalanine can have a helical structure. In addition, they suggested piling a double layer of polyphenyl-alanine on a gold surface so that the polyalanine can have a helical structure, since the polyphenyl-alanine has too small a space to form a helical structure (Science 261, 73 (1993)). Even though the method in which protein is introduced by changing the surface may be applied to DNA, a bigger dendrimer should be used since a double helix of the protein has a bigger diameter than that of DNA (A type is 25.5 Å and B type is 23.7 Å). The dendrimer is applied only to gold, because the dendrimer comprises sulfur.

### SUMMARY OF THE INVENTION

It is an object of the present invention to provide a compound used for preparation of a substrate having a molecular layer comprising amine groups with a low density on the surface, and a method of preparing the same.

It is another object to provide a substrate including a molecular layer having amine groups with a low density and with a certain distance between the amine groups on the surface, and a method of preparing the same.

It is another object to provide a method of forming a molecular film with a stable structure through multiple ionic bonds on the surface.

It is another object to provide a substrate having desired molecules fixed easily on the substrate surface, since the substrate comprises a molecular layer with a certain distance between amine groups.

It is another object to provide a substrate that is applicable to preparation of DNA chips and bio-chips, where fixation of desired molecules on its surface, and study of the surface is easy, and a method of preparing the same.

These objects may be achieved by a compound that is represented by Chemical Formula 1:

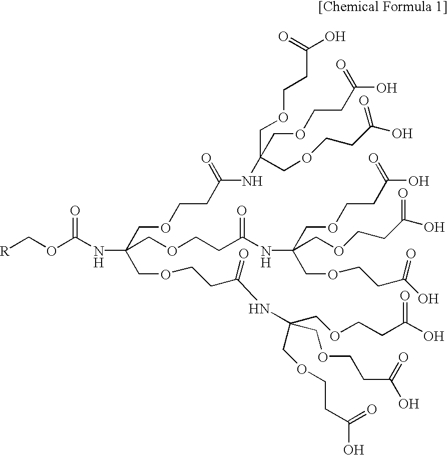

wherein R includes phenyl; phenyl substituted with nitro, halogen or cyano group; naphthyl; or anthryl.

These objects may also be achieved by a preparation method of the compound represented by Chemical Formula 1 in a preparation method of a carboxylic derivative represented by Chemical Formula 1, which comprises:

a) cyanoethylation of tris(hydroxymethyl)aminomethane and acrylonitrile to prepare tris[(cyanoethoxy)methyl]aminomethane;

b) refluxing of the tris[(cyanoethoxy)methyl]aminomethane added with concentrated hydrochloric acid in order to prepare tris[(carboxyethoxy)ethyl]methyl]aminomethane;

c) esterifcation of the tris[(carboxyethoxy)ethyl]methyl] aminomethane by addition of methanol in order to prepare tris[[(methoxycarbonyl)ethoxy]methyl]aminomethane;

d) protecting of the tris[(methoxycarbonyl)ethoxy]methyl]aminomethane by addition of a compound represented by Chemical Formula 2 in order to prepare a compound represented by Chemical Formula 3:

[Chemical Formula 2]

ROCOCl

wherein the R includes phenyl; phenyl substituted with nitro, halogen, or cyano group; naphthyl; or anthryl,

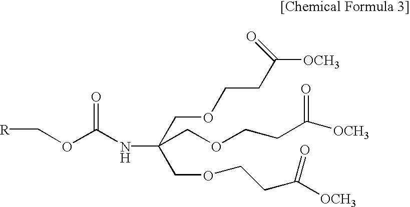

wherein the R includes phenyl; phenyl substituted with nitro, halogen, or cyano group; naphthyl; or anthryl;

e) hydrolyzing the mixture after adding sodium hydroxide solution to the compound represented by Chemical Formula 3 in order to prepare a compound represented by Chemical Formula 4:

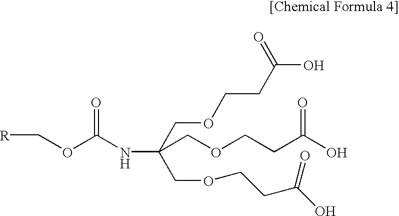

wherein the R includes phenyl; phenyl substituted with nitro, halogen, or cyano group; naphthyl; or anthryl,

f) reacting the mixture after dissolving the compound represented by Chemical Formula 4 and the tris[(methoxycarbonyl)ethoxy]methyl]aminomethane in dimethylformamide (DMF) and adding dicyclohexylcarbodiimide (DCC) and hydroxybenzotriazole (HOBT) to the dissolved material in order to prepare a compound represented by Chemical Formula 5:

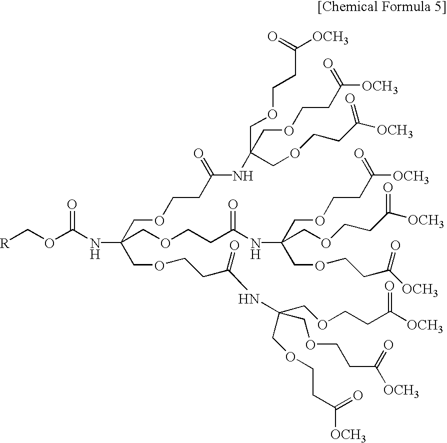

wherein the R includes phenyl; phenyl substituted with nitro, halogen, or cyano group; naphthyl; or anthryl;

g) hydrolyzing the mixture after adding sodium hydroxide solution to the compound represented by Chemical Formula 5 in order to prepare the compound represented by Chemical Formula 1.

These objects may also be achieved by a substrate having a molecular layer prepared by reaction of amine groups on the surface of an aminosilylated substitute and a derivative compound having a carboxylic acid represented by Chemical Formula 1 on its surface.

In addition, these objects may be achieved by a preparation method of a substrate having a molecular layer with a controlled amine density and regular spacing on its surface, which comprises:

a) a step of preparing a substrate having an amino silane layer on its surface; and

b) a step of reacting amine groups produced from the amino silane layer with a carboxylic derivative.

Preferably, the end group of the derivative of the step b) includes both the carboxylic acid and the amine group, and more preferably, the derivative includes the compound represented by Chemical Formula 1.

### BRIEF DESCRIPTION OF THE DRAWINGS

A more complete appreciation of the invention, and many of the attendant advantages thereof, will be readily apparent as the same becomes better understood by reference to the following detailed description when considered in conjunction with the accompanying drawings, wherein:

FIG. 1 is a schematic diagram illustrating a preparation procedure for a substrate with a controlled amine density and regular spacing with use of a compound represented by Chemical Formula la of the present invention;

FIG. 2 is a schematic diagram in series with FIG. 1 illustrating a preparation procedure for a substrate with a controlled amine density and regular spacing with use of a compound represented by Chemical Formula 1a;

FIG. 3 is a graph showing a stability of the substrate of Example 2 relative to various pH levels;

FIG. 4 is a graph showing a stability of the substrate of Example 2 relative to temperature;

FIG. 5 is a graph showing stability of the substrate of Example 3 relative to temperature;

FIG. 6 is a graph showing stability of the substrate of Example 3 relative to various pH levels;

FIG. 7 is an atomic force microscope (AFM) image analysis showing a CBZ group not-deprotected by neat trifluoroacetic acid;

FIG. 8 is an AFM image analysis showing a CBZ group deprotected by neat trifluoroacetic acid;

FIG. 9 is UV-visible spectrum showing a molecular layer of a compound represented by Chemical Formula 1a, wherein “a” shows a CBZ group after deprotection, “b” shows formed 9-antraldehydroimine, and “c” is after hydrolysis;

FIG. 10 is a fluorescence spectrum at concentrations of 20, 40, 60, 80, and 100 mM, wherein the insert represents a calibration curve showing the relation of fluorescence intensity versus 9-anthraldehyde.

### DETAILED DESCRIPTION AND THE INVENTION

In the following detailed description, only the preferred embodiment of the invention has been shown and described, simply by way of illustration of the best mode contemplated by the inventors of carrying out the invention. As will be realized, the invention is capable of modification in various obvious respects, all without departing from the invention. Accordingly, the drawings and description are to be regarded as illustrative in nature, and not restrictive.

In the present invention, a substrate, on which a molecular layer having amine groups with desirable and regular spacing is formed, is prepared by reacting amine groups produced from a surface layer of an aminosilylated substrate with a carboxylic derivative. In particular, the molecular layer having amine groups with a certain spacing is formed by preparing a polymer derivative represented by Chemical Formula 1 with a regular molecular weight, and the polymer is a cone-shaped and hyperbranched molecule having one amine group and nine carboxylic groups. That is, the molecular layer having amine groups with a low density and a certain spacing is formed by reacting the amine groups produced from an aminosilylated substrate with the polymer represented by Chemical Formula 1.

In order to prepare the substrate of the present invention, the compound represented by Chemical Formula 1 is prepared first. In the chemical formula, R includes phenyl; phenyl substituted with a nitro, halogen or cyano group; naphthyl, or anthryl. In detail, the R includes phenyl of a benzene ring; 2-nitrobenzyl, 3-nitrobenzyl, 4-nitrobenzyl, 2-fluorobenzyl, 3-fluorobenzyl, 4-fluorobenzyl, 2-chlorobenzyl, 3-chlorobenzyl, 4-chlorobenzyl, 2-bromobenzyl, 3-bromobenzyl, 4-bromobenzyl, 2-iodobenzyl, 3-iodobenzyl, 4-iodobenzyl, 2-cyanobenzyl, 3-cyanobenzyl, or 4-cyanobenzyl, that is substituted with a electron-withdrawing group; or 1 -naphthyl, 2-naphthyl, or 9-anthryl, which is modified from a benzene ring.

The preparation procedure of the compound represented by Chemical Formula 1 is shown in Reaction 1 and Reaction 2:

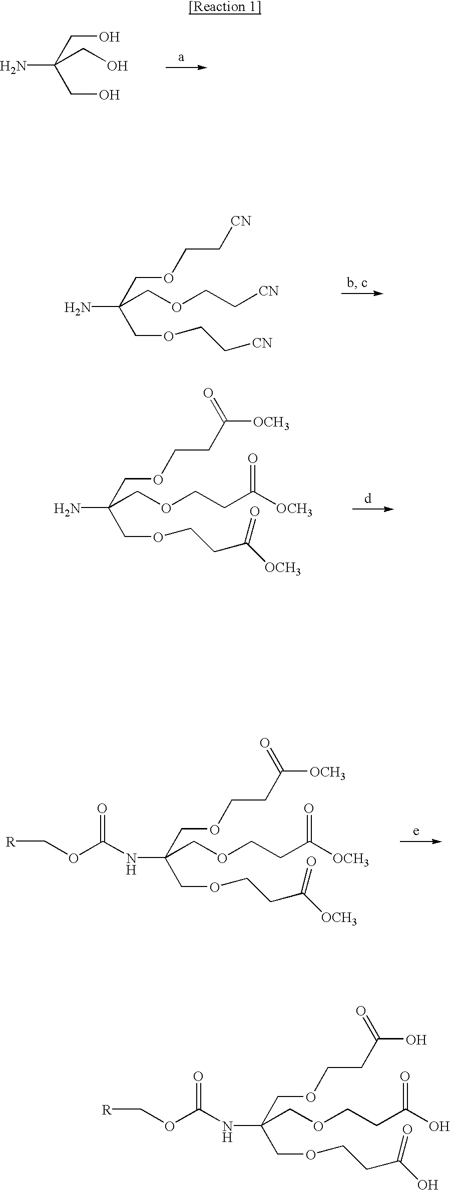

wherein,

a is step of adding CH₂═CHCN, KOH, and p-dioxane at 25° C. for 48 hours,

b is the step of adding concentrated hydrochloric acid to the mixture and refluxing the solution for 3 hours,

c is the step of adding MeOH to the solution and stirring the resulting mixture at 25° C. for 24 hours,

d is the step of adding the compound represented by Chemical Formula 2, NaHCO₃, and H₂O to the stirred mixture and further reacting the mixture at 25° C. for 12 hours,

e is the step of adding 1 N NaOH to the mixture of d at 25° C. for 12 hours, and

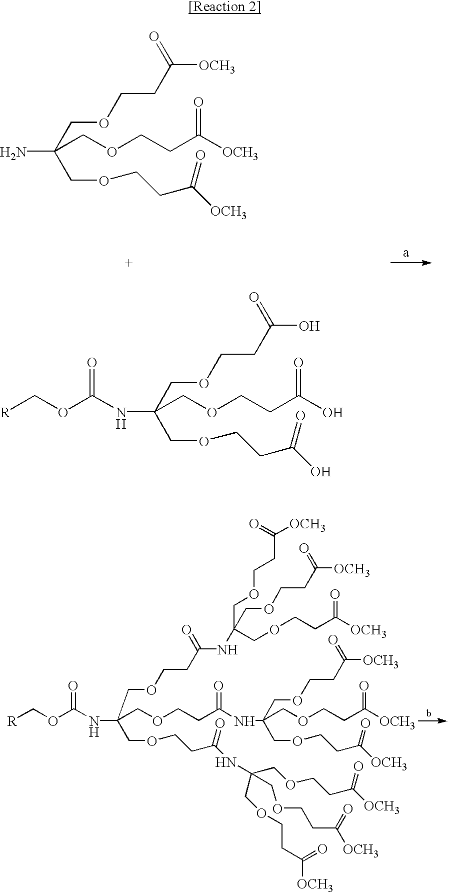

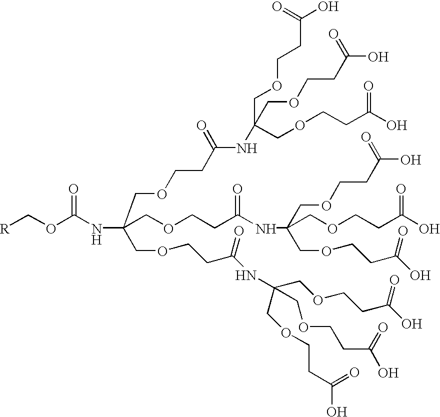

wherein,

a is the step of adding DCC, 1-hydroxybenzotriazole, and DMF, and reacting the mixture at 25° C. for 48 hours,

b is the step of adding 1 N NaOH to the mixture of a, and reacting the resulting mixture at 25° C. for 12 hours.

A representative compound among the compounds represented by Chemical Formula 1 is N-(benzyloxycarbonyl)-tris[((N′-(carbonyl)-tris((carboxyethoxy)methyl)methylamino)ethoxy)methyl]aminomethane (hereinafter referred to as N—CBZ-[1]amine-[9] acid) of Chemical Formula 1a, wherein the R is phenyl.

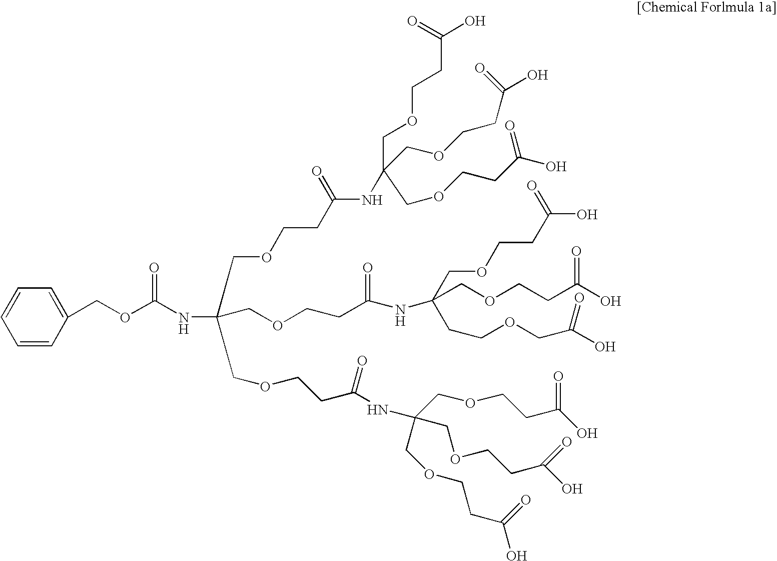

Hereinafter, the N—CBZ-[1]amine-[9] acid is regarded as a representative compound among the compounds represented by Chemical Formula 1. Among the compounds represented by Chemical Formula 1, the other compounds except N—CBZ-[1]amine-[9] acid represented by, Chemical Formula 1a are prepared in the same manner as the N—CBZ-[1]amine-[9] acid except for use of a raw material, and the properties of the compounds are similar to those of the N—CBZ-[1]amine-[9] acid.

In the present invention, the amine groups produced from the molecular layer formed on the substrate surface reacts with the compound represented by Chemical Formula 1, such as N—CBZ-[1]amine-[9] acid, so that the amine density decreases by the desirable amount. The end group of the N—CBZ-[1]amine-[9] acid is protected by CBZ (carbobenzyloxy).

The N—CBZ-[1]amine-[9] acid having a protected amine group is designed such that the amine group is not transformed by reactants during reaction on the substrate surface, so the unneeded reaction does not occur. The CBZ deprotects easily, and is capable of turning into the primary amine group.

In the preparation of N—CBZ-[1]amine-[9] acid represented by Chemical Formula 1a, the repeating unit of the N—CBZ-[]amine-[9] acid is critical, and it is prepared with tris(hydroxymethyl)aminomethane which is commonly used and is inexpensive, as a raw material. The other compounds represented by Chemical Formula 1 except the N—CBZ-[1]amine-[9] acid are prepared in the same manner as the N—CBZ-[1]amine-[9] acid.

The method developed by Bruson is followed for preparation of tris[(cyanoethoxy)methyl]aminomethane through cyanoethylation of tris(hydroxymethyl)aminomethane and acrylonitrile. The tris(hydroxymethyl)aminomethane and potassium hydroxide should be dried sufficiently under a vacuum for use in the preparation, since the tris(hydroxymethyl)aminomethane and potassium hydroxide are hygroscopic. In the preparation of tris[(cyanoethoxy)methyl]aminomethane, the amount of potassium hydroxide is critical. The amount of potassium hydroxide ranges from 5 to 20 wt % based on the amount of tris(hydroxymethyl)aminomethane, and is preferably 15 wt %. When the amount of potassium hydroxide is greater than 20 wt % the acrylonitrile may be excessively polymerized, but when the amount of potassium hydroxide is less than 5 wt % the cyanoethylation of tris(hydroxymethyl)aminomethane and acrylonitrile may not occur. After completion of the reaction, a specific nitrile peak appeared at 118.5 ppm of ¹³C NMR, and the peak is identical to the spectrum of the compound prepared by Newkome.

In order to obtain tris[(carboxyethoxy)methyl]aminomethane, tris[(cyanoethoxy)methyl]aminomethane is refluxed in hydrochloric acid for 3 hours, though the tris[(cyanoethoxy)methyl]aminomethane is separated through column chromatography, since it is soluble in organic solvents. The nitrile group is changed to carboxylic acid, and a large amount of NH₄Cl salt is obtained as a byproduct. After the NH₄Cl is filtered by dissolving it in acetone and vacuum-distilling it, 176.2 ppm carboxylic acid peak appears at ¹³C NMR 118.5 ppm without the specific nitrile peak. Though many kinds of protecting reagents are used in order to protect the tris[(carboxyethoxy)methyl]aminomethane, the protection fails due to the hydrogen bond of carboxylic acid and the amine groups. Therefore, the carboxylic acid of the end group is needed to protect it.

The tris[(carboxyethoxy)methyl]aminomethane is an oily and acidic compound, and the mixture is esterificated when methanol is added to it. Tris[((methoxycarbonyl)ethoxy)methyl]aminomethane is prepared simply by the esterification of the mixture, and carboxylic acid, as an end group of the tris[((methoxycarbonyl)ethoxy)methyl]aminomethane, is protected. Newkome suggested a simple method in which hydrogen chloride was injected into tris[((methoxycarbonyl)ethoxy)methyl]aminomethane added to ethanol, but the yield was low and the process procedure was dangerous due to use of hydrogen chloride. The method of the present invention is simpler than that of Newkome, and the yield is higher. Tris[((methoxycarbonyl)ethoxy)methyl]aminomethane peaks appear at 176.2 ppm and 51.6 ppm of ¹³C NMR due to the ester and methoxy groups, respectively.

A repeating unit for preparation of the dendrimer is tris[((methoxycarbonyl)ethoxy)methyl]aminomethane, and a core unit is a protected tris[((methoxycarbonyl)ethoxy)methyl]aminomethane. Di-tert-butyl dicarbonate and benzyl chloroformate are used for amine protection. The protection of two test samples was easy, but the BOC(t-butoxycarbonyl) may have a problem changing ester into carboxylic acid.

When the prepared N-(BOC)-tris[(carboxyethoxy)methyl]aminomethane is separated, it returns to tris[(carboxyethoxy)methyl]aminomethane, since the BOC in HCl solution is hydrolyzed. When the N-(BOC)-tris[carboxyethoxy]methyl]aminoethane is coupled with aqueous DCC (Dicyclohexylcarbodiimide) in order to prevent the BOC hydrolysis, the reaction does not occur at all. It is supposed that the low yield results from a typically low yield of the peptide bond used which reacts with solution DCC.

Since the CBZ that is used with benzylchloroformate is stable to HCl used in the workup, N-(benzyloxycarbonyl)-tris[(carboxyethoxy)methyl]aminomethane is easy to separate as an organic layer. The N-(benzyloxycarbonyl)-tris[(carboxyethoxy)methyl]aminomethane shows CBZ peaks at 128.7 ppm and 128.2 ppm, and a carbamate peak at 155.2 ppm.

In N—CBZ-[1]amine-[9] acid preparation, the coupling yield is lowest at 33.3%, when 4.5 N of tris[((methoxycarbonyl)ethoxy)methyl]aminomethane and N-(benzyloxycarbonyl)-tris[carboxyethoxy]methyl]aminomethane are dissolved in DMF, 3 N of DCC and HOBT are added thereto, and it is all stirred for 48 hours. As the reaction proceeds, dicyclohexylurea is generated, which is not soluble in DMF.

The N-(benzyloxycarbonyl)-tris[((N′-(carbonyl)-tris-(((methoxycarbonyl)ethoxy)methyl)methylamino)ethoxy)methyl]aminomethan e shows specific ester and amide peaks at 172.3 ppm and 171.3 ppm respectively, due to ¹³C NMR. The size of the peaks is in the ratio of 1:3. The FAB result indicates that N-(benzyloxycarbonyl)-tris[((N′-(carbonyl)-tris(((methoxycarbonyl)ethoxy)methyl)methylamino)ethoxy)methyl]aminomet han-e is prepared at 1556(M⁺+1).

N-(benzyloxycarbonyl)-tris[((N′-(carbonyl)-tris(((methoxycarbonyl)ethoxy)methyl)methylamino)ethoxy) methyl]aminomet han-e is hydrolyzed in 1 N NaOH to obtain N-(benzyloxycarbonyl)-tris[((N′-(carbonyl)-tris-((carboxyethoxy) methyl) methylamino)ethoxy)methyl] aminomethane). The N-(benzyloxycarbonyl)-tris[((N′-(carbonyl)-tris((carboxyethoxy)methyl)methylamino)ethoxy)methyl]aminomethane is mass-analyzed, and the peak appears at 1429(M⁺). Tables 1 to 6 show the IR results and analysis results of the compounds.

TABLE 1

| | |
|---|---|
| tris[(cyanoethoxy)methyl] | ¹H NMR(CDCl₃) |
| aminomethane | δ 3.68(t, CH₂CH₂CN, 6), |
| | 3.42(s, CH₂OCH₂CH₂, 6H), |
| | 2.63(t, CH₂OCH₂CH₂, 6H), |
| | 1.83(s, H₂N, 2H). ¹³C NMR(CDCl₃) |
| | δ 118.5(CH₂CH₂CN), 72.7(CH₂OCH₂CH₂), |
| | 66.1(CH₂OCH₂CH₂), 56.4(H₂NC(CH₂—)₃), |
| | 19.1(CH₂CH₂C N). |

TABLE 2

| | |
|---|---|
| tris[((methoxycarbonyl) | ¹H NMR(CDCl₃) |
| ethoxy)methyl] | δ 3.72-3.68(m, CH₂CH₂COOCH₃, 15H), |
| aminomethane | 3.34(s, CH₂OCH₂CH₂, 6H), |
| | 2.58(t, CH₂OCH₂CH₂, 6H), 1.83(s, H₂N, 2H). |
| | ¹³C NMR(CDCl₃) |
| | δ 172.1(CH₂COOCH₃), 72.6(CH₂OCH₂CH₂), |
| | 66.8(CH₂OCH₂CH₂), 56.0(H₂NC(CH₂—)₃), |
| | 51.6(CH₂COOCH₃), |
| | 34.8(CH₂COOCH₃). |
| | IR(CHCl₃) |
| | 3376, 2953, 2871, 1740, 1587, 1438, 1361, |
| | 1265, 1197, 1112, 1074, 1023 cm⁻¹. |
| | Anal. Calc'd for C₁₆H₂₉NO₉ |
| | C, 50.65; H, 7.70; N, 3.69. Found: C, 50.63; |
| | H, 7.81; N, 3.97. |

TABLE 3

| | |
|---|---|
| N-(benzyloxycarbonyl)- | ¹H NMR(CDCl₃) |
| tris[((methoxycarbonyl) | δ 7.33(m, C₆H₅CH₂, 5H), |
| ethoxy)methyl] | 5.28(s, OCONH, 1H), 5.03(s, C₆H₅CH₂O, 2H), |
| aminomethane | 3.69-3.64(m, CH₂OCH₂CH₂COOCH3, 21H), |
| | 2.52(t, CH₂OCH₂CH₂, 6H). |
| | ¹³C NMR(CDCl₃) |
| | δ 172.1(CH₂COOCH₃), 155.3(OCONH), |
| | 137.1(C₆H₅CH₂), |
| | 128.7(C₆H₅CH₂), 128.2(C₆H₅CH₂), |
| | 69.6(CH₂OCH₂CH₂), |
| | 67.0(CH₂OCH₂CH₂), 66.3(C₆H₅CH₂), |
| | 59.0(OCONHC(CH₂—)₃), |
| | 51.6(CH₂COOCH₃), 34.8(CH₂COOCH₃). |
| | IR(CHCl₃) |
| | 3379, 3027, 2952, 2879, 1738, 1509, 1438, |
| | 1363, 1235, 1199, 1112, 1072, 1027 cm⁻¹. |
| | Anal. Calc'd for C₂₄H₃₅NO₁₁ |
| | C, 56.13; H, 6.87; N, 2.73. Found: C, 56.23; |
| | H, 6.90; N, 2.88. |

TABLE 4

| | |
|---|---|
| N-(benzyloxycarbonyl)- | ¹H NMR(CDCl₃) |
| tris[(carbonylethoxy) | δ 10.00(br, CH₂COOH, 3H), |
| methyl]aminomethane | 7.32(m, C₆H₅CH₂, 5H), 5.28(s, OCONH, 1H), |
| | 5.03(s, C₆H₅CH₂O, 2H), |
| | 3.66(m, CH₂OCH₂CH₂COOH, 12H), |
| | 2.52(t, CH₂OCH₂CH₂, 6H). |
| | ¹³C NMR(CDCl₃) |
| | δ 177.5(CH₂COOH), 155.2(OCONH), |
| | 137.1(C₆H₅CH₂), 128.7(C₆H₅CH₂), |
| | 128.2(C₆H₅CH₂), 69.8(CH₂OCH₂CH₂), |
| | 66.8(CH₂OCH₂CH₂), 60.9(C₆H₅CH₂), |
| | 59.1(OCONHC(CH₂—)₃), 35.0(CH₂COOH). |
| | IR(CHCl₃) |
| | 3600-2300, 3340, 3026, 2927, 2882, 1714, |
| | 1517, 1455, 1417, 1241, 1193, 1110, |
| | 1071 cm⁻¹. |
| | Anal. Calc'd for C₂₁H₂₉NO₁₁ |
| | C, 53.50; H, 6.20; N, 2.97. Found: C, 53.49; |
| | H, 6.52; N, 2.64. |

TABLE 5

| | |
|---|---|
| N-(benzyloxycarbonyl)- | ¹H NMR(CDCl₃) |
| tris[(N′-(carbonyl)- | δ 7.32(m, C₆H₅CH₂, 5H), |
| tris-(((methoxycarbonyl) | 6.18(s, CH₂CONH, 3H), 5.64(s, OCONH, 1H), |
| ethoxy)methyl) | 5.03(s, C₆H₅CH₂O, 2H), |
| methylamino) | 3.68-3.65(m, CH₂OCH₂CH₂COOCH₃, |
| ethoxy)methyl]methyl | CH₂OCH₂CH₂CONH, 75H), |
| aminomethane | 2.52(m, CH₂OCH₂CH₂, 24H). |
| | ¹³C NMR(CDCl₃) |
| | δ 172.3(CH₂COOCH₃), 171.3(CH₂CONH), |
| | 155.2(OCONH), 137.1(C₆H₅CH₂), |
| | 128.7(C₆H₅CH₂), 128.2(C₆H₅CH₂), |
| | 69. 6(CH₂OCH₂CH₂), 67.8(C₆H₅CH₂), |
| | 67.0(CH₂OCH₂CH₂), |
| | 60.0(CH₂CONHC(CH₂—)₃), |
| | 59.2(OCONHC(CH₂—)₃, 51.9(CH₂COOCH₃), |
| | 37.6(CH₂CONH), 35.0(CH₂COOCH₃). |
| | MS(FAB⁺, m/z) 1556.2(M+1). |
| | IR(CHCl₃) |
| | 3369, 3067, 2953, 2877, 1736, 1668, 1528, |
| | 1438, 1368, 1328, 1265, 1199, 1109, |
| | 1026 cm⁻¹. |
| | Anal. Calc'd for C₆₉H₁₁₀NO₃₅ |
| | C, 53.27; H, 7.13; N, 3.60. Found: C, 53.03; |
| | H, 7.27; N, 3.78. |

TABLE 6

| | |
|---|---|
| N-(benzyloxycarbonyl)- | ¹H NMR(DMSO) |
| tris[(N′-(carbonyl)- | δ 12-10(br, CH₂COOH, 9H), |
| tris-((carboxyethoxy)- | 7.37(m, C₆H₅CH₂, 5H), |
| methyl)methylamino)- | 7.09(s, CH₂CONH, 3H), 6.27(s, OCONH, 1H), |
| ethoxy)methyl] | 5.02(s, C₆H₅CH₂O, 2H), |
| aminomethane | 3.71-3.60(m, CH₂OCH₂CH₂COOH, |
| | CH₂OCH₂CH₂CONH, 48H), |
| | 2.45(m, CH₂OCH₂CH₂, 24H). |
| | ¹³C NMR(DMSO) |
| | δ 173.2(CH₂COOH), 171.0(CH₂CONH), |
| | 155.2(OCONH), 137.1(C₆H₅CH₂), |
| | 128.7(C₆H₅CH₂), 128.1 (C₆H₅CH₂), |
| | 68.7(CH₂OCH₂CH₂), 67.9(C₆H₅CH₂), |
| | 67.2(CH₂OCH₂CH₂), |
| | 60.3(CH₂CONHC(CH₂—)₃), |
| | 60.2(OCONHC(CH₂—)₃, |
| | 37.6(CH₂CONH), 35.0(CH₂COOCH₃). |
| | MS(FAB⁺, m/z) 1429.6(M+). |
| | IR(neat) |
| | 3600-2300, 3342, 3026, 2924, 2880, 1715, |
| | 1651, 1528, 1455, 1417, 1196, 1109 cm⁻¹. |
| | Anal. Calc'd for C₆₀H₉₂NO₃₅ |
| | C, 49.18; H, 6.60; N, 3.82. Found: C, 49.32; |
| | H, 6.84; N, 3.64. |

Hereinafter, a formation method of molecular layers having a controlled amine density on the surface is described, wherein a compound represented by Chemical Formula 1 is capable of self-assembly and self-attachment on the substrate surface.

A substrate surface is cleaned and dried. The dried substrate is immersed in a solution of aminosilane compound and a solvent for a predetermined time in order to aminosilylate the substrate. The aminosilane compound may be a compound that does not produce an acidic byproduct, such as 3-aminopropyl-tri-ethoxysilane, 3-aminopropyl-di-ethoxymethyl silane, and 3-aminopropylethoxy-di-methyl silane, and the solvent includes toluene that dissolves the aminosilane compound. The substrate includes silicon wafer, glass, silica and fused silica. After completion of the aminosilylation, the substrate is cleaned with the solvent and dried, and the resultant substrate having an aminosilylated surface is immersed in a solvent comprising N—CBZ-[1]amine-[9] acid under an inert atmosphere at a room temperature for 12 hours, for dissolution.

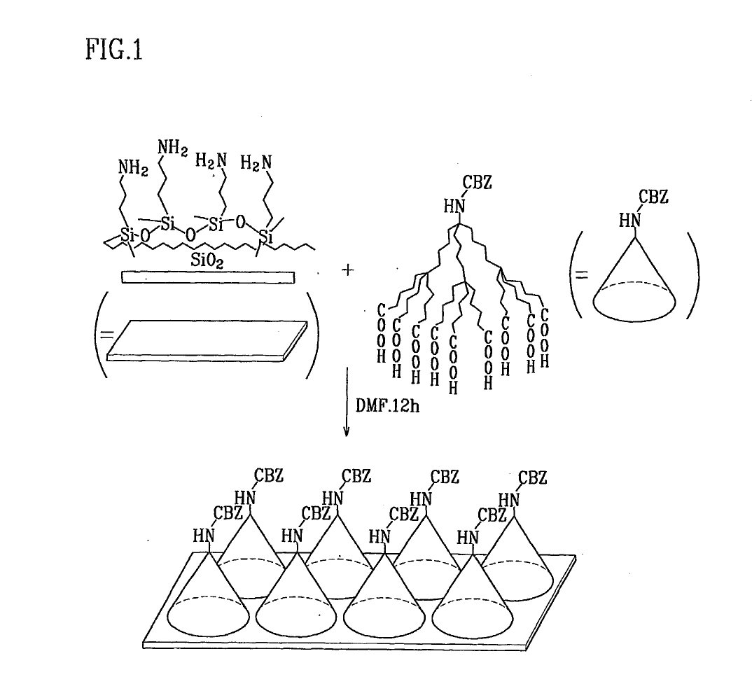

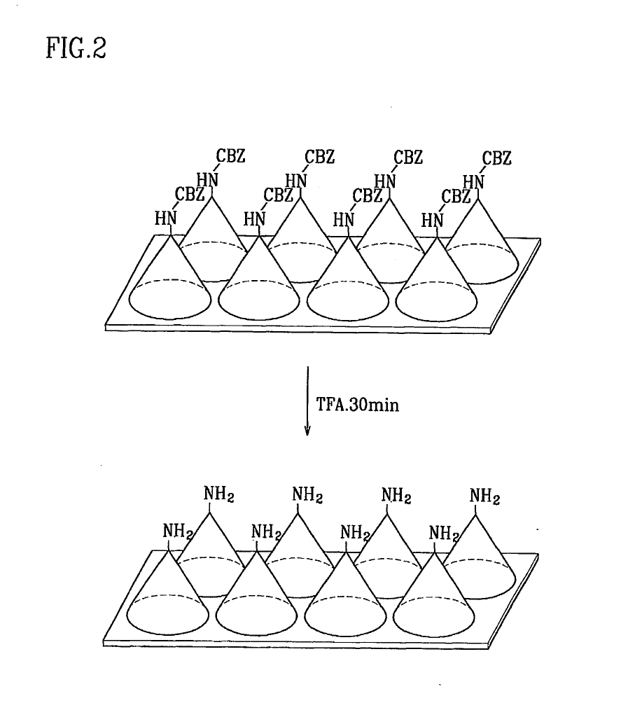

Since the amine group in the end group of the N—CBZ-[1]amine-[9] acid is protected, a protecting group should be removed from the N—CBZ-[1]amine-[9] acid in order to reveal the amine group on the substrate surface. The substrate is subjected to dissolution in trifluoroacetic acid and is sonicated at room temperature so that the amine group is deprotected. Then, the substrate surface is cleaned with a copious amount of solvent such as methanol, and the trifluoroacetic acid and the separated protecting group, which are adsorbed physically on the surface, are removed from the surface. In FIG. 1 and FIG. 2, a substrate having a molecular layer obtained by the aforementioned procedure is represented.

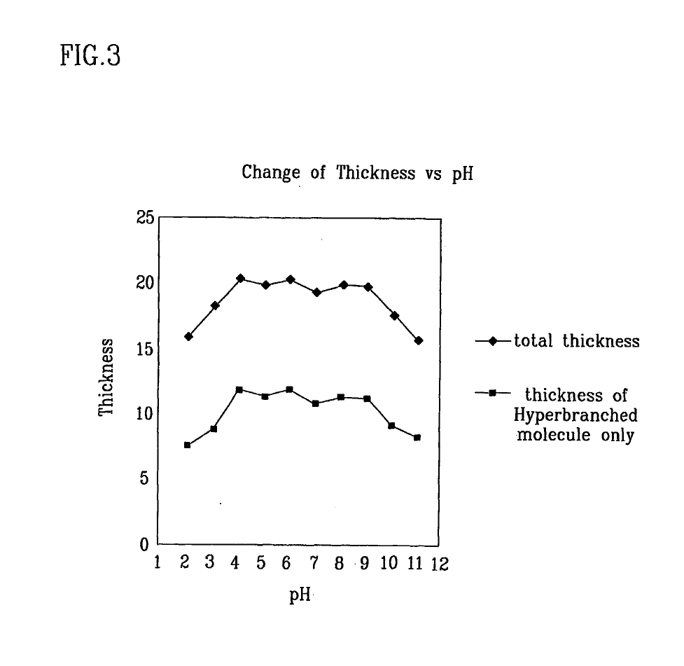

In FIG. 1, a carboxylic acid in the end group of the N—CBZ-[1]amine-[9] acid is strongly ionic-bonded with the amine on the aminosilylated substrate surface, and the carboxylic acid is strongly adsorbed on the aminosilylated substrate surface. In FIG. 3, the bond stability of the carboxylic acid and the aminosilylated substrate surface is represented. The bond is stable at various pH levels, and in particular the bond is so stable at a neutral pH that the molecular thin layer is useful. In FIG. 2, the protected amine groups on the surface of the substrate are all changed to primary amines, and the primary amines have a strong reactivity.

The amine density of the solid substrate with a desired amine density according to the present invention ranges from 0.05 to 0.3 amines/nm², so the solid substrate may be applied to and take an important role in preparation of DNA chips and Bio-chips since each amine is distributed uniformly on the substrate surface.

For example, when an oligonucleotide is fixed on a solid substrate in order to prepare a DNA chip, and the solid substrate has a controlled amine density, the controlled density decreases steric hindrance between bio-molecules. Therefore, the DNA chip has a high stability and is easily prepared.

In addition, when the amine density is controlled in order to fix enzymes or other bio-molecules on a substrate surface, the enzymes or other bio-molecules are fixed on the substrate surface easily, and the chip yield increases. Desired molecules may be fixed on a substrate surface by the procedure of the present invention, and the surface may be studied. Therefore, sufficient spacing made by a method of the present invention enables each bio-molecule to work as an effective sensor.

The following examples illustrate the present invention in further detail, but the present invention is not limited by these examples.

### EXAMPLE 1

1) Preparation of Tris[(cyanoethoxy)methyl]aminomethane

A round-bottomed flask containing tris(hydroxymethyl)aminomethane (20.2 g, 167 mmol) and potassium hydroxide (3.0 g, 53 mmol) was placed under vacuum for 12 hours. After dissolving the solids in p-dioxane (500 ml), 3.5 equivalent of acrylonitrile (38.5 ml, 585 mmol) was added dropwise with the aid of a syringe pump. After stirring for another 24 hours at room temperature, the reaction was quenched by adding copious amount of chloroform. Washing with water, drying the organic solution with anhydrous MgSO₄, and evaporation of the solvents resulted in the crude liquid product. The crude liquid was loaded in a column packed with silica gel and eluted for the purification (eluent; ethyl acetate: methanol=4:1 (v/v), R_{f}; 0.64). Total weight of the final yellow liquid was 34.8 g and the yield of Tris[(cyanoethoxy)methyl]amino methane was 74.3%.

2) Preparation of Tris[((methoxycarbonyl)ethoxy)methyl]aminomethane

Tris[(cyanoethoxy)methyl]aminomethane (2.0 g, 7.1 mmol) was placed in a 500 ml round-bottomed flask, and 20 ml of hydrochloric acid was added thereto. After heating the mixture to reflux it for 3 hours, the acid was removed with a rotary evaporator. An efficient cold trap filled with potassium hydroxide was placed between the evaporator and a water pump to avoid hydrochloric acid contamination of the machinery system and the environment. The evaporation resulted in a brownish viscous liquid. After the removal of the acid, 200 ml of acetone was added to dissolve the residue while the flask was warm. The resulting solution was filtered to remove NH₄Cl. The filtrate was evaporated to dryness, and redissolved in 200 into CHCl₃. Drying the solution with anhydrous MgSO₄ and evaporation of the solvent resulted in a crude oil. The crude oil was loaded in a column packed with silica gel, and eluted (eluent: ethyl acetate:methanol=8:1 (v/v), R_{f}; 0.25) in order to obtain a pure yellow liquid. The total weight of the yellow liquid was 2.33 g, and the yield was 80.6%.

3) Preparation of N-(Benzyloxycarbonyl)-tris[((methoxycarbonyl)ethoxy)methyl]aminomethane

Tris[((methoxycarbonyl)ethoxy)methyl]aminomethane (1.0 g, 2.5 mmol) was dissolved in 10 ml water, and the aqueous solution was cooled to 0° C. with the use of an ice bath. 0.3 g sodium hydrogen carbonate was added, and the resulting solution was stirred at 0° C. for 1 hour. Benzyl chloroformate (0.50 ml, 3.5 mmol) was slowly added while the solution was further stirred at 0° C., and the temperature of the solution was raised to room temperature after the addition. In 24 hours, an immiscible organic layer was formed, and the organic layer was extracted with 50 ml ethyl acetate. The organic solution was treated with anhydrous MgSO₄, and filtered. Evaporation to dryness gave the crude product. The product was loaded in a column packed with silica gel, and eluted (eluent: ethyl acetate:hexane=1:1 (v/v), R_{f}; 0.46) to obtain a pure yellow oil. The total weight of the yellow oil was 1.01 g, and the yield was 77.3%.

4) Preparation of N-(Benzyloxycarbonyl)-tris[(carboxyethoxy)methyl]aminomethane

N-(Benzyloxycarbonyl)-tris[((methoxycarbonyl)ethoxy)methyl]aminomethane (2.0 g, 3.7 mmol) was dissolved in 5 ml methanol, and an excess amount of 1.0 N sodium hydroxide (15 ml, 150 mmol) was added while stirring. At the beginning of the addition, the solution turned milky, but it eventually became clear. After stirring at room temperature for 12 hours, the solution was evaporated to dryness, and 50 Ml of water was subsequently added. The aqueous solution was extracted with CHCl₃ to remove the unhydrolyzed ester. The flask containing the resulting aqueous layer was placed in an ice bath, and the solution was acidified with dilute hydrochloric acid at a temperature of between 0 and 5° C. The best result was obtained when the pH level of the solution was adjusted to be around 1.5. After the aqueous solution was saturated with sodium chloride, the product was extracted with 200 Ml of ethyl acetate. The organic layer was dried with anhydrous MgSO₄, and the solvent was removed with the aid of a rotary evaporator. The crude product was loaded in a column packed with silica gel. Elution through the column (eluent: ethyl acetate:methanol=2:1 (v/v), R_{f}; 0.72) resulted in a viscous yellow liquid. The total weight of the yellow liquid was 1.52 g, and the yield was 82.4%.

5) Preparation of N-(Benzyloxycarbonyl)-tris[((N′-(carbonyl)-tris(((methoxycarbonyl)-ethoxy)methyl)methylamino)ethoxy)methyl]aminomethane

Triacid N-(Benzyl oxycarbonyl)-tris[(carboxyethoxy)methyl]aminomethane (1.37 g, 2.9 mmol), 3 equivalent of dicyclohexylcarbodiimide (DCC; 1.77 g, 8.66 mmol), and 3 equivalent of 1 -hydroxybenzotriazole (HOBT; 1.17 g, 8.66 mmol) were added together, and the resulting solution was stirred for 48 hours at room temperature. It was observed that an insoluble white solid formed upon the reaction. The solution was evaporated to dryness, and the residue was redissolved in 100 Ml dichloromethane. The solution was filtered to remove insoluble solids, and dried with anhydrous MgSO₄. The crude product obtained after evaporation of the solvent was loaded in a column packed with silica gel, and eluted (eluent: ethyl acetate:methanol=4:1 (v/v), R_{f}; 0.82) to obtain a highly viscous yellow liquid. The total weight of the highly viscous yellow liquid was 1.50 g, and the yield was 33.3%.

6) Preparation of N-(Benzyloxycarbonyl)-tris[((N′-(carbonyl)-tris((carboxyethoxy)methyl)methylamino)ethoxy) methyl]aminomethane

N-(Benzyloxycarbonyl)-tris[((N′-(carbonyl)-tris(((methoxycarbonyl)-ethoxy)methoxy)methylamino)ethoxy)methyl]aminomethane (2.00 9, 1.28 mmol) was dissolved in 5 Ml methanol, and an excess amount of 1.0 N NaOH (15 Ml, 150 mmol) was added while stirring. At the beginning of the addition, the solution turned milky, but it eventually became clear. After stirring for 24 hours, the solution was evaporated to dryness. 50 Ml of water was added to the residue, and the aqueous solution was shaken with CHCl₃ to remove the unhydrolyzed starting material. The resulting aqueous layer was acidified with dilute hydrochloric acid of which pH ranges from 1 to 2 at a temperature of between 0 to 5° C. The best result was obtained when the pH level of the solution reached one or two. After adding 2.0 g NaCl to the solution, the product was extracted with 200 Ml ethyl acetate in the acidic condition. Evaporation of the organic solvent resulted in a viscous yellow liquid. The total weight of the viscous yellow liquid was 1.34 g, and the yield was 73.3%.

### EXAMPLE 2

A cleaned silica substrate was dried under a 20 mTorr vacuum.

3-(aminopropyl)diethoxymethylsilane dissolved in 10⁻³ M toluene was placed in a round-bottomed flask under a nitrogen atmosphere, and the dried silica substrate was added thereto in order to aminosilylate the substrate.

The aminosilylated substrate was cleaned with toluene, and dried in an oven at 120° C. for 30 minutes. The dried substrate was cooled to room temperature, dissolved in a sequence of toluene, a mixture of toluene and methanol in the volume ratio of 1:1, and methanol, and the dissolved substrate was sonicated for 3 minutes. The sonicated substrate was dried under a 20 mTorr vacuum, and was dissolved in the solvent comprising N—CBZ-[1]amine-[9] acid prepared in Example 1 under an inert atmosphere. The resulting substrate was reacted at room temperature for 12 hours.

The reacted substrate was dissolved in a sequence of methanol, a mixture of methanol and water in a volume ratio of 1:1, water, and methanol, and the dissolved substrate was sonicated for 3 minutes, and dried under vacuum.

The silica substrate was dissolved in trifluoroacetic acid in order to remove the CBZ group from the silica substrate, and was sonicated at room temperature for 30 minutes. After sonication, the substrate was cleaned with a copious amount of methanol, and was further sonicated for 10 minutes.

The layer thickness of aminosilane molecule and the surface density of amines before and after reaction with the N—CBZ-[1]amine-[9] acid were measured. The initial layer of aminosilane molecules was about 8 Å in thickness, and the initial surface density of amines was 3.5 amines/nm².

The layer thickness of the aminosilane layer after reacting with N—CBZ-[1]amine-[9] acid increased by approximately 10 Å, to 18 to 19 Å, and the surface density of the amine group decreased by 0.18 amines/nm². During measurement of the surface density of the amine, 9-anthraldehyde, which is 6 times greater in water absorptivity than the conventional one, was used, since calculation of the 4-nitrobenzaldehyde was not possible due to the substantial decrease of the surface density of the amines.

In addition, the surface structure was observed with an Atomic Force Microscope (AFM), and was found to be similar to that of the aminosilylated substrate. That is, a single layer of hyperbranched molecules and a uniform molecular layer were formed on the substrate.

The stability of the thin film having N—CBZ-[1]amine-[9] acid was measured by sonicating the thin film in deionized water in time terms of 30 minutes, 1 hour, 2 hours, 4 hours, and the measured thickness at each time was not changed. When the thin film was allowed to stand in deionized water for 24 hours and 48 hours, the measured thickness at each time was not changed.

FIG. 3 shows stability of the thin film in water according to various pH levels. In a pH range of 4 to 9, the thin film shows itself to be stable, but below a pH level of 3 and the above a pH level of 9, the thickness of the thin film decreases substantially. Therefore, it is shown that the thin film is stable in a wide pH range as well as in physiological acidity.

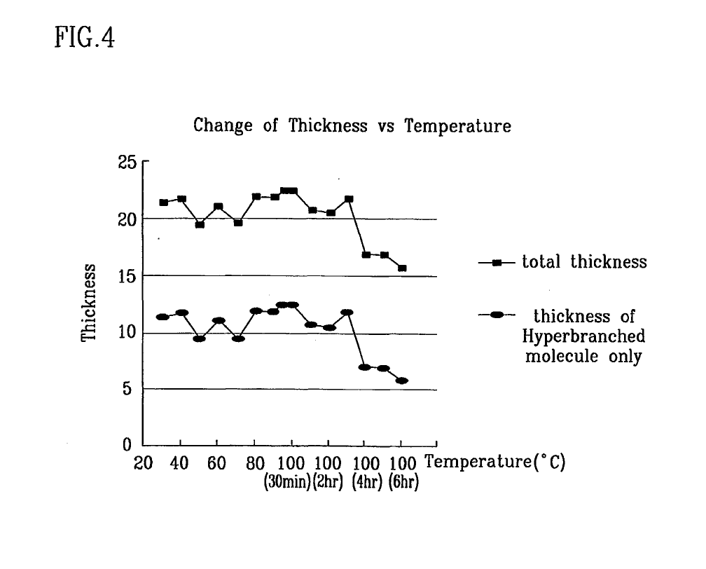

The thin film was stable in water at high temperatures. FIG. 4 shows the thickness of the thin film according to varying temperature. When the thin film was soaked in solution for 30 minutes while varying the temperature between 40° C. and 100° C. in increments of 10° C., the thickness in the test was not changed. When the thin film was soaked in water at 100° C. for more than 4 hours, the thickness of the thin film decreased. Therefore, the thin film has thermal stability, and may be applied to bio-chips.

The unit of thickness is A in FIGS. 3 and 4.

### EXAMPLE 3

A substrate was treated in the same manner as in Example 2, except that a fused silica substrate was used instead of a silica substrate. The fused silica substrate has similar characteristics to those of the silica substrate in Example 2.

The characteristics of the fused silica was measured as follows:

(Aminosilylation)

A two-neck round bottom 250 ml flask was evacuated with a vacuum pump, and nitrogen was allowed to fill the flask. Subsequently, anhydrous toluene (20 ml) and a coupling agent (0.2 ml) were added through a septum. Clean substrates were placed into the solution for 3 hours. Typically, no more than four substrates were placed in a flask in order to avoid a physical overlap among the substrates. After the self-assembly, the substrates were taken out of the flask, washed with toluene, and placed in glass vials. The vials were placed in an oven, and heated at 110° C. for 30 minutes. The plates were immersed in toluene, toluene-methanol (1:1 in the volume ratio), and methanol in a sequential manner, and they were sonicated for 3 minutes at each washing step. Each washed plate was placed in a vial, and several of such vials were placed in a glass container with a large screw cap lined with an O-ring, and eventually the container was evacuated under pressure ranging from 30 to 40 Torr, for dryness.

(Self-Assembly of “Nonapus”, N—CBZ-[1]amine-[9] Acid)

A two-neck round bottom 250 ml flask was evacuated with a vacuum pump, and nitrogen was allowed to fill the flask. A certain amount of N—CBZ-[1]amine-[9] acid was dissolved in a mixed solvent of dimethylformamide (DMF) and deionized water in a volume ratio of 1:1 to prepare a 20 ml solution. The solution was added to the nitrogen-filled flask, and subsequently some of the above prepared aminosilylated substrates were placed in the solution. The flask was allowed to stand at room temperature for the self-assembly, and each of the substrates was taken out of the solution after a certain lapse of time. Right after being taken out, the plates were washed with a copious amount of deionized water. Each substrate was sonicated for 3 minutes in deionized water, a mixture of deionized water-methanol in the volume ratio of 1:1, and methanol in a sequential manner. After the sonication, each substrate was placed in a vial, and the several of such vials were placed in a glass container with a large screw cap lined with an O-ring, and eventually the container was evacuated under pressure ranging from 30 to 40 Torr, for dryness.

(Quenching of Residual Amines by Acetic Anhydride)

Residual amines that are not ionic-bonded with N—CBZ-[1]amine-[9] acid are preferably treated with acetic anhydride to remove the reactivity of the residual amines, since residual amines inhibit the deprotection.

A flask containing 4-(dimethylamino)pyridine (1 mg, 8.2 μmol) was evacuated, and nitrogen was allowed to fill the flask. Subsequently, anhydrous methylene chloride (20 ml) and acetic anhydride (1 ml, 11 mmol) were added through a septum. Several of the above self-assembled substrates were placed in the solution at room temperature for 12 hours. After the reaction, each plate was taken out of the flask, washed with methylene chloride, and placed in a glass vial. The vials were sonicated for 3 minutes at each step while they were filled with methylene chloride, methanol, and methylene chloride in a sequential manner. After the sonication, each of the substrates was placed in a vial, and the several of such vials were placed in a glass container with a large screw cap lined with an O-ring, and eventually the container was evacuated under pressure ranging from 30 to 40 mTorr, for dryness.

(Thickness and Absorption)

When the aminosilylated substrate was self-assembled with nonacarboxylic acid, the static water contact angle marginally decreased from 70(±2)° to 65(±2)°. Even the smaller contact angle does not seem to reflect the hydrophobicity of the phenyl group at the top of the molecular layer. If we had a compact molecular layer that had a hydrophobic tail group, a large increase of water contact angle should have been observed. The observation is in harmony with the characteristic molecular structure of the N—CBZ-[1]amine-[9] acid, because the cone shape of the molecule allows water to reach polar parts of the self-assembled layer including the amide and the ether group, and maybe even the carboxylic acid and the protonated amine. Therefore, the phenyl of CBZ protecting group did not increase the water contact angle upon self-assembly.

As fused silica substrates are transparent to UV-like rays, the assembly was monitored with a spectrophotometer. A 0.001(±0.0003) increase of absorption at 260 nm was observed upon the self-assembly, and this increase value was significant in considering the precision of the instrument. While λₘₐₓ of N—CBZ-[1]amine-[9] acid in methanol is 258 nm, the absorption of the acid on the surface is rather broad. It is not clear why the absorption band is broadened even if it is believed that the phenyl chromophores are separated from each other by a distance (ca. 20˜30 Å) sufficient to avoid the mutual interaction. After being treated with the acetic anhydride, the absorption band of the substrate was not changed. The constancy means that the assembled nonacarboxylic acid was not desorbed from the aminosilylated surface when it was treated with acetic anhydride.

Thickness of the aminosilylated substrate was 8(±2) Å, and that was increased to 18(±2) Å after being self-assembled with the N—CBZ-[1]amine-[9] acid.

(pH-Stability of the Self-Assembled Molecular Layer)

After self-assembly of N—CBZ-[1]amine-[9] acid, the plates were placed in a vial containing water of various pH levels ranging from 2.0 to 11.0. The pH of the solution was adjusted by adding an appropriate amount of 0.1 N NaOH and 0.1 N HCl, or a mixture thereof in a suitable amount. Typically, one or two plates were put in a vial of a particular pH. After leaving vials at room temperature for 3 hours, the plates were taken out of the solution, and washed with deionized water. The plates were sonicated for 3 minutes while they were immersed in deionized water and methanol in a volume ratio of 1:1, and methanol in a sequential manner. After the sonication, each piece of the substrates was placed in a vial, and several of such vials were placed in a glass container with a large screw cap lined with an O-ring, and eventually the container was evacuated under pressure ranging from 30 to 40 mTorr, for dryness. After quenching of the residual amines with acetic anhydride, pH-stability of the capped molecular layer was also investigated in the same manner.

Since the ionic interaction between the carboxylic acid and primary amine is utilized on the assembly, the attractive force will fail at both a strong acidic and a basic condition in which the carboxylate group is protonated and the RNH₃⁺ group is deprotonated, respectively. Given that the Kₐ of acetic acid and RNH₃⁺ are 1.8×10⁻⁵ and ca. 10^{-10.5,} respectively, the number of ions of the self-assembled molecule or the aminosilylated surface will be reduced by half at a pH of 4.7 to 10.5. The reduction of ion density is expected to reduce the attraction force significantly.

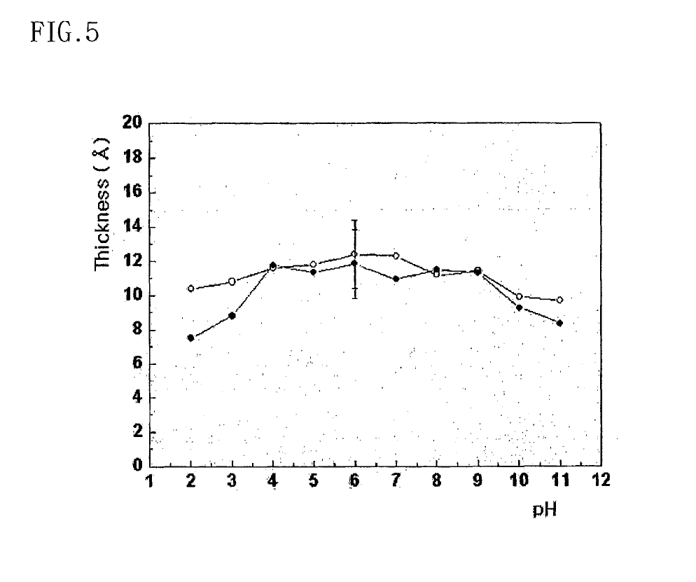

The thickness of the molecular layer was measured after washing the plates with deionized water and subsequently with methanol. In FIG. 5, the symbol (●) indicates the thickness of the nonacarboxylic acid layer before and the symbol (∘) indicates that after treatment with acetic anhydride. As is obvious from FIG. 5, the molecular layer is stable at pH levels ranging from 4 to 9. Considering the pKₐ involved, the ionic interaction is not strong enough to keep the molecular layer intact as soon as the carboxylic acid starts to protonate or the RNH₃⁺ group begins to deprotonate. However, the pH-stability window seems to be wide enough for most biomedical applications.

(Thermal Stability of Self-Assembled Molecular Layer in Water)

After the self-assembly of the N—CBZ-[1]amine-[9] acid, the plates were soaked in a test tube containing deionized water. The test tube was placed in an oil-bath with a controllable temperature, and heated to a predetermined temperature for 30 minutes. At 100° C., the heating time increases incrementally. When the water evaporated, more was added in order to maintain a certain water level. The plate was cleaned with deionized water, further cleaned with methanol, and then evacuated under a pressure ranging from 30 to 40 mTorr. The pH stability of the capped molecules was tested in the same manner of the aforementioned, after the reactivity of acetic anhydrous residual amines was removed.

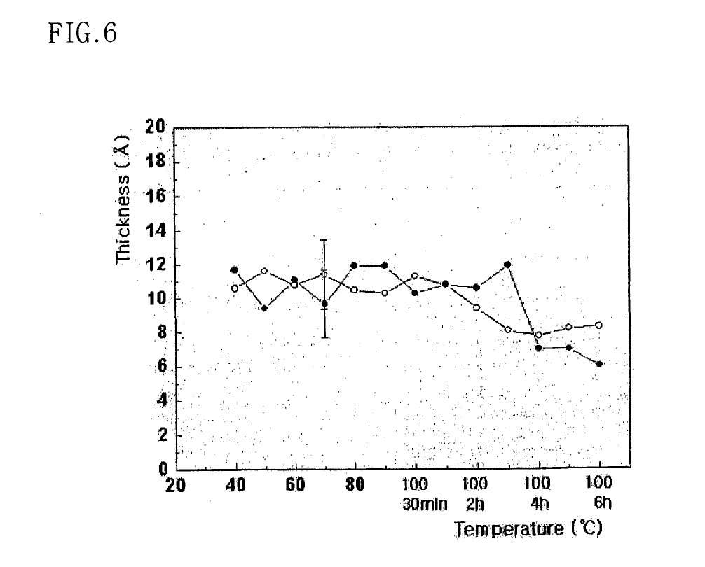

Thermal stability of the molecular layer in deionized water was examined at various temperatures. After the substrates were heated at the particular temperature for 30 minutes, the thickness of the organic film was measured. In FIG. 6, the solid circle (●) and open circle (∘) indicates the thickness of the nonacarboxylic acid molecular layer before and after being treated with acetic anhydride, respectively. Both molecular layers were stable up to 100° C., and only an extended heating time at 100° C. reduced the thickness. For example, heating at 100° C. for 6 hours decreased the thickness by 6 Å. In this experiment it was found that the molecular layer formed through the 9-point ionic interaction is strong enough to survive heating at 100° C. for 1 to 2 hours in water.

(Deprotection of CBZ from the Self-Assembled Hyperbranched Carboxylic Acid)

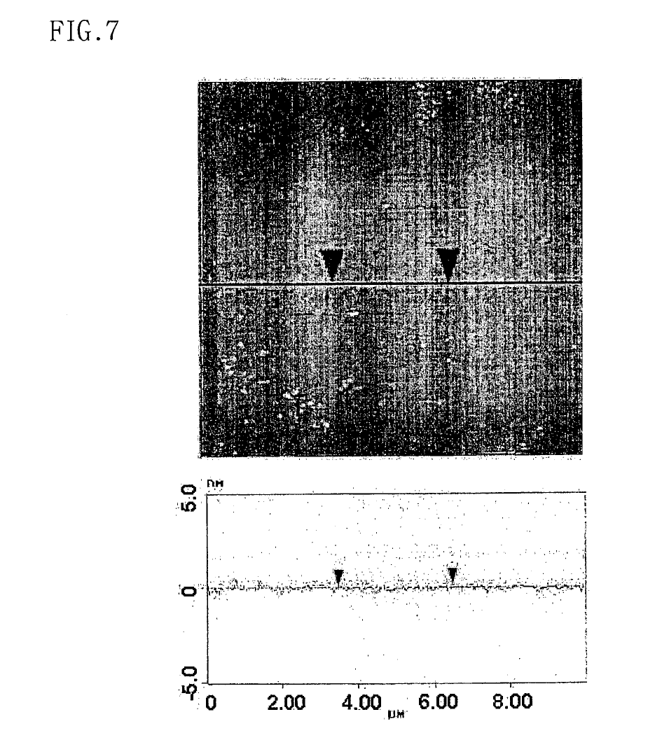

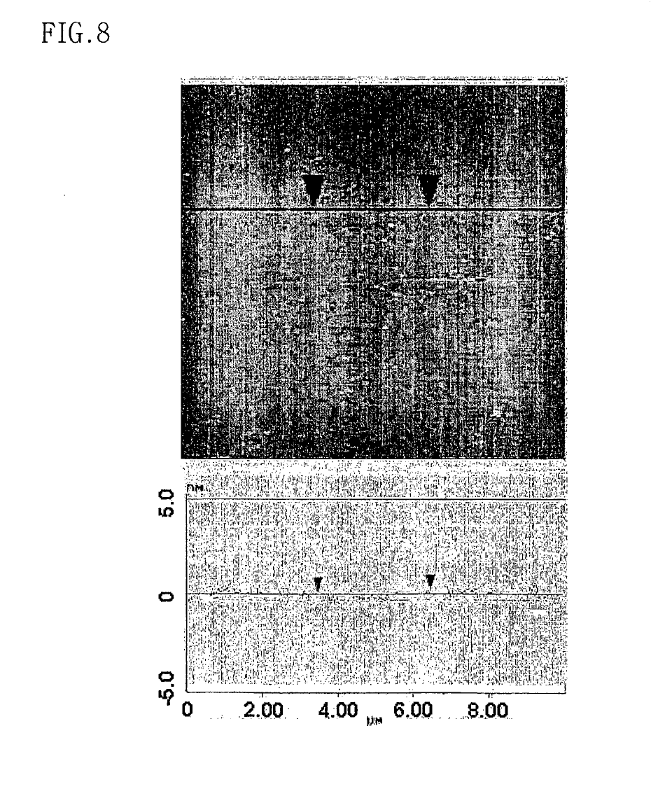

It is important to deprotect CBZ for the further application, while the condition should be mild enough to keep the molecules intact on the surface. It was observed that soaking the substrates in neat trifluoroacetic acid for 30 minutes at room temperature was effective to deprotect the CBZ protecting group. Spectroscopic analyses confirmed that the nonacarboxylic acid stayed on the surface under the particular condition. As illustrated earlier (FIGS. 7 and 8), the self-assembled molecules would have desorbed from the surface if the substrates were left longer in the acidic environment. Upon the deprotection, the thickness of the organic layer decreased typically by 2 to 3 Å, and the AFM image (FIG. 7) and absorption spectrum after deprotection were almost unchanged. Therefore, it is demonstrated that trifluoroacetic acid hydrolyzes the CBZ protecting group to generate the reactive primary amine on the top of the hyperbranched molecule without tampering with the self-assembled body itself. (Surface density of the primary amines)

<Imine Formation>

A two-neck round bottom 250 ml flask containing 9-anthraldehyde (10 mg, 48 μ mol) was evacuated, and nitrogen was allowed to fill the flask. Subsequently, anhydrous ethanol (20 ml) was added through a septum. The substrate under investigation was placed in the solution at 50° C. for 12 hours. Typically, 2 to 4 substrates were employed for the test. After the imine formation, the plates were taken out of the solution and washed with methanol. Subsequently, the plates were placed in a beaker having methanol therein, and sonicated for 3 minutes. After repeating the last sonication process three times-with fresh solvent, each plate was placed in a vial, and a glass container holding the resulting vials was evacuated under a pressure ranging from 30 to 40 mTorr for dryness.

<Hydrolysis>

The imine-formed substrates were placed in a 10 ml test tube containing 3 ml of deionized water at 50° C. for 18 hours. After the hydrolysis, the plates were taken out of the solution, and the accurate volume of the solution and the intensity of fluorescence of 9-anthraldehyde were measured.

As established in this laboratory, 4-nitrobenzaldehyde has been utilized to measure the surface density of free primary amines on the surface of a substrate. A molar absorptivity (1.45×10⁴ L/cm·mol) of 4-nitrobenzenaldehyde is sufficient for the accurate detection of a density as high as 1.0 amines/nm². For a lower density, molecules with a larger molar absorptivity should be utilized. In this regard, 9-anthraldehyde with a molar absorptivity is 8.70×10⁴ L/cm·mol qualifies. As the density reduces to below 1.0 amines/nm², a larger space occupied by the anthracene moiety would not be problematic for the full derivatization of the amine under the investigation.

For the control reaction, the imine formation of 9-anthraldehyde was tested for the aminosilylated surface. The imine formation in anhydrous solvent and hydrolysis in water were successful under the typical condition. However, because of a larger space occupied by the anthracene moiety, the measured surface density of free primary amines on the aminosilylated layer was lower than that measured with slim 4-nitrobenzaldehyde. It was also observed that over 95% of the surface amines were blocked by the acetic anhydride in the employed condition. For another control reaction, the imine formation with 9-anthraldehyde was tested for the CBZ-protected N—CBZ-[1]amine-[9] acid molecular layer before and after treatment with acetic anhydride. A measurable degree of the imines at the uncapped substrate indicated that there were still free amine groups available for imine formation, while spectroscopically the imines were not found in the capped substrate. Therefore, this control experiment shows that all the active primary amine groups remaining on the surface were capped by treating with acetic anhydride.

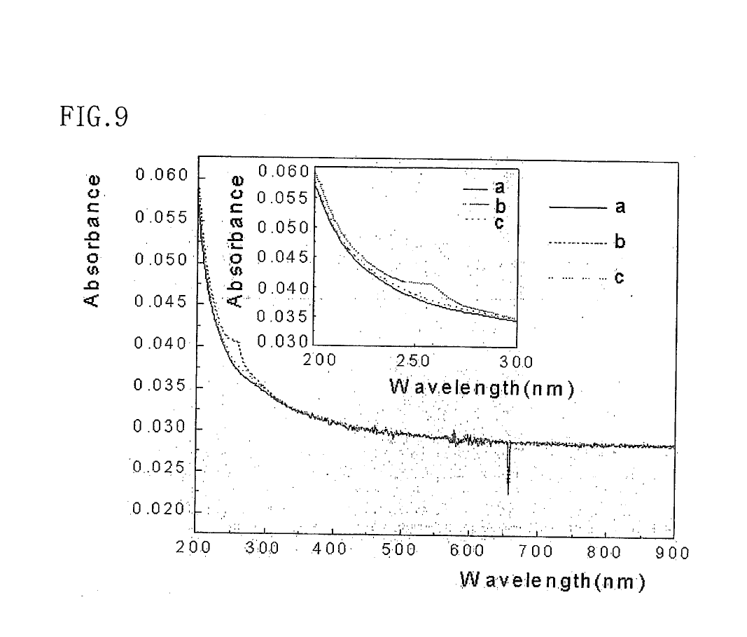

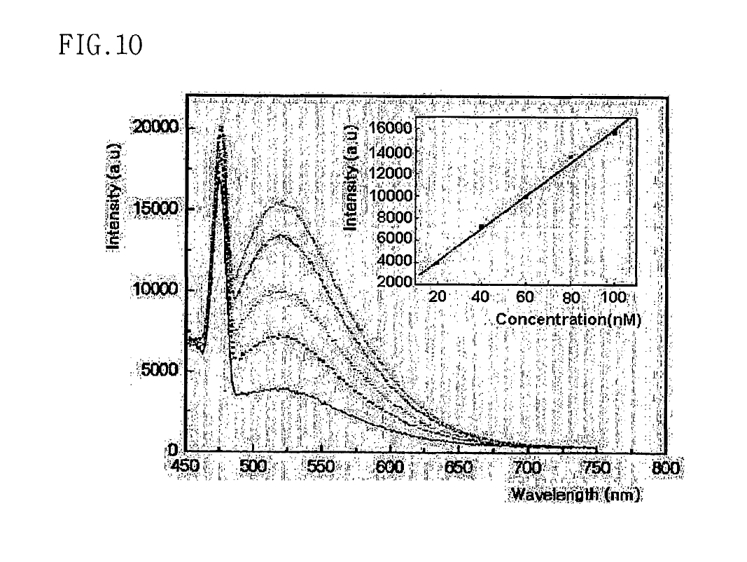

Finally, the 9-anthraldehyde on the surface was treated with tri-fluoroacetic acid so that the CBZ group was deprotected. FIG. 9 shows the peaks appearing at 259 nm due to imine formation. The imine-derived substrate surface was impregnated with deionized water in order to hydrolyze the substrate, and 9-anthraldehyde was prepared by hydrolysis of the imines. A fluorescent 9-anthraldehyde was further used in order to confirm the calculation. The calibration curve in FIG. 10 shows that the fluorescent 9-anthraldehyde is proportional to the concentration. Measuring the fluorescence of the fluorophore revealed a surface density of 0.1 to 0.25 amines/nm². In other words, each hyperbranched molecule occupies area in the range of 4 to 10 nm².

According to the present invention, the amine density of the substrate surface is capable of being controlled and decreased, allowing the solid substrate having the controlled amine density to take an important role in development of DNA chips and biochips. In addition, a film according to the present invention is stable at various pH levels and high temperatures, and the stability results from a multiple bond among 9 carboxylic acids and the substrate surface. Compared to a single bond and a 3-point bond that are not stable, a 9-point bond is highly stable. The film is used for fixation of desired molecules on a substrate, and for a surface substrate in studies of surface characteristics.

While the present invention has been described in detail with reference to the preferred embodiments, those skilled in the art will appreciate that various modifications and substitutions can be made thereto without departing from the spirit and scope of the present invention as set forth in the appended claims.

What is claimed is:

1. A compound represented by Chemical Formula 1: 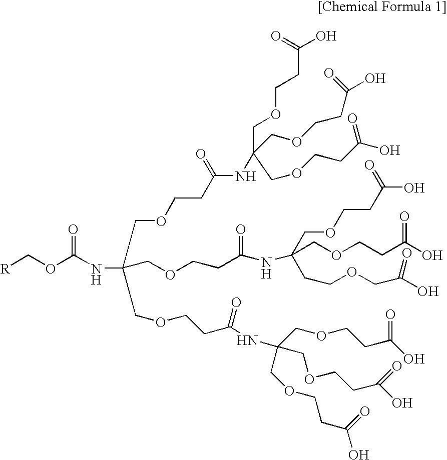
wherein R is phenyl; phenyl substituted with nitro, halogen or cyano group; naphthyl; or anthryl.

2. The compound of claim 1, wherein the compound is N—CBZ-[1]amine-[9]acid represented by Chemical Formula 1a:

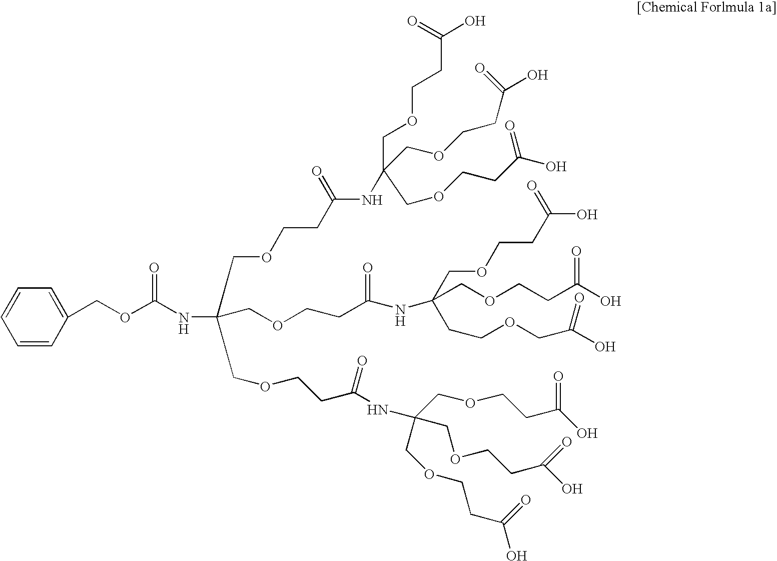

3. A preparation method of the carboxylic derivative represented by Chemical Formula 1, which comprises: 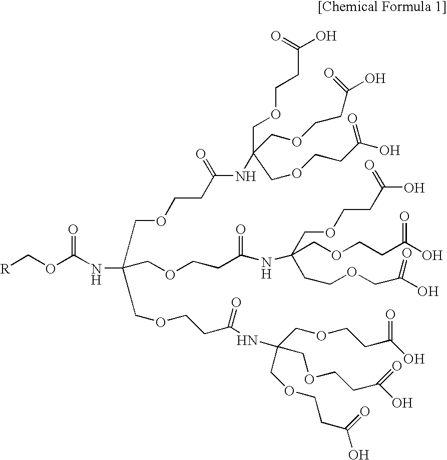
wherein R is phenyl; phenyl substituted with nitro, halogen, or cyano group; naphthyl, or anthryl:
a) a step of cyanoethylating tris(hydroxymethyl)aminomethane and acrylonitrile in order to prepare tris[(cyanoethoxy)methyl]aminomethane;
b) a step of refluxing the mixture, after adding concentrated hydrochloric acid to the tris[(cyanoethoxy)methyl]aminomethane in order to prepare tris[(carboxyethoxy)ethyl]methyl]aminomethane;
c) a step of esterificating the tris[(carboxyethoxy)ethyl]methyl]aminomethane by adding methanol to the tris[(carboxyethoxy)ethyl]methyl]aminomethane in order to prepare tris[((methoxycarbonyl)ethoxy)methyl]aminomethane;
d) a step of protecting the tris[((methoxycarbonyl)ethoxy)methyl]aminomethane by adding a compound represented by Chemical Formula 2 to the tris[((methoxycarbonyl)ethoxy)methyl]aminomethane in order to prepare a compound represented by Chemical Formula 3:
ROCOCI Chemical Formula 2]
wherein R is phenyl; phenyl substituted with nitro, halogen or cyano group; naphthyl; or anthryl, 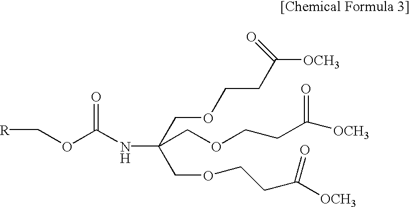
wherein R is phenyl; phenyl substituted with nitro, halogen, or cyano group; naphthyl; or anthryl;
e) a step of hydrolyzing the mixture after adding sodium hydroxide solution to the compound represented by Chemical Formula 3 in order to prepare a compound represented by Chemical Formula 4: 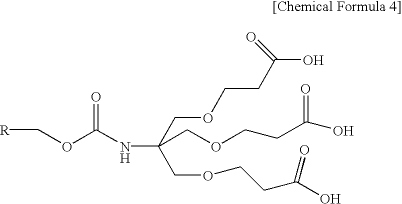
wherein R is phenyl; phenyl substituted with nitro, halogen or cyano group; naphthyl; or anthryl;
f) a step of reacting the mixture after dissolving the compound represented by Chemical Formula 4 and the tris[((methoxycarbonyl)ethoxy)methyl]aminomethane in dimethylformamide (DMF) and adding dicyclohexylcarbodiimide and hydroxybenzotriazole to the dissolved material to prepare a compound represented by Chemical Formula 5: 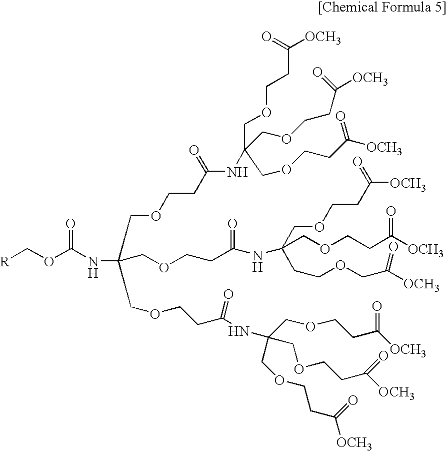
wherein R is phenyl; phenyl substituted with nitro, halogen, or cyano group; naphthyl; or anthryl; and
g) a step of hydrolyzing the mixture after adding sodium hydroxide solution to the compound represented by Chemical Formula 5 in order to prepare the compound represented by Chemical Formula 1.

4. The method according to claim 3, wherein the compound of the step d) is benzylchloroformate.

5. A substrate comprising a molecular layer prepared by reacting amine groups on the surface of an aminosilylated substrate with a cone-shaped carboxylic derivative represented by Chemical Formula 1 on the substrate surface: 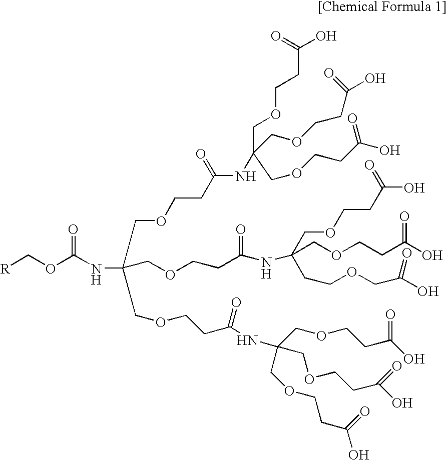
wherein R is phenyl; phenyl substituted with nitro, halogen, or cyano group; naphthyl; or anthryl.

6. The substrate according to claim 5, wherein the amine density of the substrate surface ranges from 0.05 to 0.3 amines/nm².

7. A preparation method of a substrate comprising a molecular layer with a controlled amine density and regular spacing, which comprises:
a) a step of preparing a substrate comprising a molecular layer of aminosilane on the substrate surface; and
b) a step of reacting the amine of the molecular layer with a carboxylic derivative.

8. The method according to claim 7, wherein the derivative of the step b) comprises both the carboxylic acid and an amine group.

9. The method according to claim 7 or claim 8, wherein the derivative of step b) is the compound represented by Chemical Formula 1: 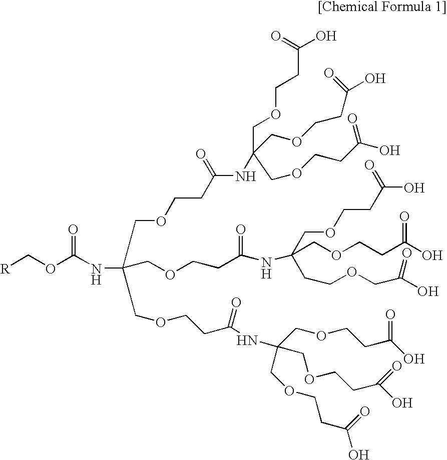
wherein R is phenyl; phenyl substituted with nitro, halogen, or cyano group; naphthyl; or anthryl.

10. The method according to claim 9, wherein the derivative is N—CBZ-[1]amine-[9]acid represented by Chemical Formula 1a:

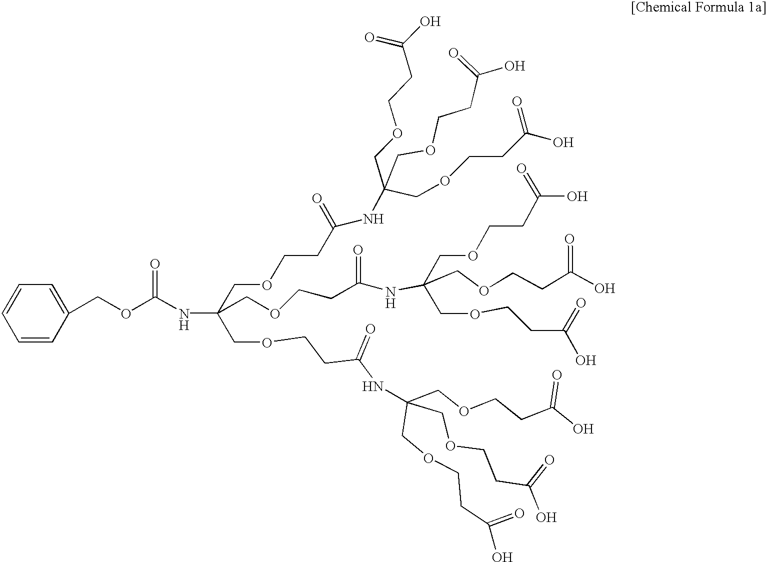

11. The method according to claim 7, wherein step b) comprises forming a thin film through an ionic bond between the derivative and the aminosilylated substrate surface.

12. The method according to claim 7, wherein the reaction in step b) is performed under an inert atmosphere.

13. The method according to claim 7, wherein the step c) comprises deprotecting the derivative by adding tri-fluoroacetic acid to the surface layer of the substrate.

14. The method according to claim 7, wherein the amine density on the substrate surface ranges from 0.05 to 0.3 amines/nm².

15. The method according to claim 7, wherein the substrate of step a) is selected from a group consisting of silicon wafer, glass, silica and fused silica.
